(19) United States
(12) Patent Application Publication
(10) Pub. No.: US 2016/0243216 A1
(43) Pub. Date: Aug. 25, 2016

(54) CMV IMMUNO-STIMULATORY COMPOSITION

(71) Applicant: THE UNIVERSITY OF SYDNEY, New South Wales (AU)

(72) Inventors: Barry Slobedman, New South Wales (AU); Simone Forbes, New South Wales (AU)

(21) Appl. No.: 15/033,267

(22) PCT Filed: Oct. 28, 2014
(86) PCT No.: PCT/AU2014/050318

§ 371 Date: Apr. 29, 2016

Related U.S. Application Data
(60) Provisional application No. 61/897,117, filed on Oct. 29, 2013.

(51) Int. Cl. A61K 39/245; C07K 14/565; A61K 39/39; C12N 7/00

(52) U.S. Cl. CPC A61K 39/245; C12N 7/00; C07K 14/565; A61K 39/39; C12N 2710/16121; C12N 2710/16151; C12N 2710/16162; C12N 2710/16134; A61K 2039/53

(57) ABSTRACT

The invention relates to a CMV strain comprising interferon bets (IFNb) useful in immuno-stimulatory compositions and vaccines

### FIELD OF THE INVENTION

The invention relates to CMV immuno-stimulatory compositions and vaccines.

### BACKGROUND OF THE INVENTION

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

A primary infection with human cytomegalovirus (HCMV) is generally asymptomatic in immuno-competent individuals however is associated with high levels of morbidity and mortality in immuno-compromised individuals.

The development of a vaccine against HCMV has been assigned the highest priority by the National Institute of Medicine, USA based on the economic cost and human suffering that would be alleviated as a result of the reduction of HCMV disease¹.

Interferon-beta (IFN-β), which exerts its effects in an autocrine and paracrine manner, has several important antiviral properties including the induction of interferon stimulated genes (ISGs) that act to block viral replication and limit viral spread.

The expression of IFN-β by a recombinant vaccinia virus was shown to limit spread of virus from an infected cell. However, the virus was still able to induce a strong CD8⁺ T-cell response, which is a hallmark of a live-attenuated vaccine². One significant finding of the study was that the limited spread of virus was observed in IFN-IIR^{-/-} mice, confirming that for vaccinia, presence of endogenous IFN-γ is not required to block replication, or otherwise to inhibit spread of virus from an infected cell.

In contrast to vaccinia, several studies have shown that the replication of HCMV is blocked by the co-activation of the IFN-IR and IFN-IIR signalling pathways³. Therefore it is has been understood that both Type I IFNs (for example, various forms of IFN-α and IFN-β) and the Type II interferon (IFN-γ) would generally be required for there to be an effective response to CMV infection. It is not known whether when the IFN-IR and IFN-IIR signalling pathways are co-activated, the CMV genome would be translated sufficiently to enable presentation of CMV peptides for the induction of the immune response.

CMV is highly virulent and rapidly spreads to bystander cells adjacent to an initially infected cell. This limits the extent to which CMV can be used as a live vaccine in prophylactic or therapeutic applications. Live vaccines are particularly preferred over peptide vaccines, as the former will generate a polyclonal response. The proportion of responders in a population to a live vaccine is likely to be much greater than the proportion of responders to a peptide vaccine, especially a peptide vaccine that has one or only a few epitopes. As an example, a phase 2 study of a CMV-vaccine indicated an efficacy of 50%; thus the protection provided was limited and a number of subjects contracted CMV infection despite the vaccination. In one case also congenital CMV was encountered.

There is a need to be able to limit the spread of CMV from an initially infected cell as this would enable CMV to be used as an immuno-stimulatory composition or vaccine capable of producing a polyclonal response across a wide proportion of the population.

### SUMMARY OF THE INVENTION

The invention seeks to address one or more of the above mentioned needs and in one embodiment provides a nucleic acid including:
a first region encoding:
   a cytomegalovirus (CMV); or
   a viral particle including a CMV protein;
a second region encoding a cytokine for preventing a CMV or viral particle translated from the first region from infecting a cell.

In another embodiment there is provided a vector including a nucleic acid as described above.

In another embodiment there is provided a virus or infective particle including a nucleic acid as described above.

In another embodiment there is provided a cell including a nucleic acid, vector, virus or infective particle as described above. The cell may further include a gene for blocking activation of an IFN-I or IFN-II signalling pathway.

In another embodiment there is provided a composition including a nucleic acid, vector, virus, infective particle or cell as described above.

In another embodiment there is provided a method for treating an individual having a CMV infection including the step of:
administering a nucleic acid, vector, virus or infective particle as described above to an individual having a CMV infection, thereby treating the individual for CMV infection.

In another embodiment there is provided a method for preventing an individual from infection with CMV including the step of:
administering a nucleic acid, vector, virus or infective particle as described above to an individual in whom CMV infection is to be prevented, thereby treating the individual for CMV infection.

In another embodiment there is provided a use of a nucleic acid, vector, virus or infective particle as described above for the treatment or prevention of CMV infection.

In another embodiment there is provided a use of a nucleic acid, vector, virus or infective particle as described above in the manufacture of a medicament for the treatment or prevention of CMV infection.

In another embodiment there is provided a nucleic acid, vector, virus or infective particle as described above for use in the treatment or prevention of CMV infection.

### BRIEF DESCRIPTION OF THE DRAWINGS

FIG. 1: Schematic diagram of the construction of Merlin HCMV IFN-β. Human IFN-β (hIFN-β) was inserted into the HCMV strain Merlin between the viral genes US15 and US16 using bacterial artificial chromosome technology.

FIG. 2: Production of IFN-β protein by Merlin HCMV IFN-β. Human foreskin fibroblasts (HFFs) were either mock infected or infected with Wild Type HCMV, HCMV IFN-β or HCMV Rescuant Virus at an MOI of 1. Supernatants were collected at 24 and 48 hours post infection (P.I.) and analysed using an IFN-β ELISA (PBL InterferonSource) to measure secreted IFN-β protein concentration.

FIG. 3: Growth Characteristics of Merlin HCMV IFN-β in Human Foreskin Fibroblasts (HFFs). Permissive HFFs were infected at an MOI of 0.01 with either HCMV Wild Type, HCMV IFN-β or HCMV Rescuant. A representative viral plaque from each infection was imaged Day 9 post infection using phase contrast/fluorescent microscopy. Permissive HFFs were either mock infected or infected at an MOI of 0.01 with either WT HCMV, HCMV IFN-j or HCMV Rescuant. Cells were collected every 3 days and virus spread was assessed by flow cytometry.

FIG. 4: Growth Characteristics of Merlin HCMV IFN-β in HFFs that express the PIV-5 V protein. HFFs that expressed the PIV-5 V protein were infected at an MOI of 0.01 with either HCMV WT, HCMV IFN-β or HCMV Rescuant. A representative viral plaque from each infection was imaged Day 9 post infection using phase contrast/fluorescent microscopy.

FIG. 5: mRNA expression of 2′,5′,-OAS1 in infected HFFs. HFFs were either mock infected or infected with HCMV Wild Type, HCMV IFN-β or HCMV Rescuant virus at an MOI of 1. Cells were harvested 24 hours post infection and cellular 2′,5′,-OAS1 mRNA was analysed using qRT-PCR. Results were normalized to GAPDH.

FIG. 6: Antiviral Properties of IFN-β produced by Merlin HCMV IFN-β. Virus-free supernatant harvested from a prior infection of HFFs was incubated with permissive HFFs before infecting the cells at an MOI of 5 with HCMV Wild Type. A representative image was taken 3 days post infection using phase contrast microscopy. An IFN-β neutralising antibody was added to the virus-free supernatant from (a) prior to its incubation with permissive HFFs. The cells were subsequently infected with HCMV Wild-Type. A representative image was taken using phase contrast microscopy before the cells were harvested and viral spread assessed using flow cytometry 3 days post infection. Note: Only the results using HCMV IFN-β are shown.

FIG. 7A: Schematic Diagram of HCMV Merlin (or HCMV Wild Type). The HCMV genome is composed of a Unique Long (UL) and Unique Short (US) Region, both flanked by inverted repeat regions (RL and RS). These are depicted by the green and orange boxes, respectively. Each of the genomic regions that underwent subsequent mutation are depicted by red boxes and expanded to highlight the ORFs (shown as arrows) located in the region. The purple arrows depict the ORFs that were deleted during recombination of other modified HCMV viruses and the grey arrows highlight the ORFs flanking the region of recombination. In the first region, the ORF UL111A is flanked by the ORFs UL105 and UL112. In the second region, the genomic region encompassing ORFs US2, US3, US6, US7, US8, US9, US10 and US11, denoted by US2-11, is flanked by the ORFs US1 and US12. In the third region the ORFs US15 and US16 are depicted. The direction of the arrows demonstrates the direction of gene transcription.

FIG. 7B: Schematic Diagram of HCMV Merlin Expressing IFNβ (or HCMV-β). The HCMV genome is composed of a Unique Long (UL) and Unique Short (US) Region, both flanked by inverted repeat regions (RL and RS). These are depicted by the green and orange boxes, respectively. Each of the genomic regions that underwent subsequent mutation are depicted by red boxes and expanded to highlight the ORFs (shown as arrows) located in the region. The purple arrows depict the ORFs that were deleted during recombination of other modified HCMV viruses and the grey arrows highlight the ORFs flanking the region of recombination. In the first region, the ORF UL111A is flanked by the ORFs UL105 and UL112. In the second region, the genomic region encompassing ORFs US2, US3, US6, US7, US8, US9, US10 and US11, denoted by US2-11, is flanked by the ORFs US1 and US12. In the third region the ORFs US15 and US16 are depicted. The insertion of the human IFN-D cDNA Cassette, using bacterial artificial chromosome (BAC) technology, between ORFs US15 and US16 is depicted using a blue arrow. The direction of the arrows demonstrates the direction of gene transcription.

FIG. 7C: Schematic Diagram of HCMV Merlin UL111A Knock-Out (or HCMV ΔUL111A). The HCMV genome is composed of a Unique Long (UL) and Unique Short (US) Region, both flanked by inverted repeat regions (RL and RS). These are depicted by the green and orange boxes, respectively. Each of the genomic regions that underwent subsequent mutation are depicted by red boxes and expanded to highlight the ORFs (shown as arrows) located in the region The purple arrows depict the ORFs that were deleted during recombination of other modified HCMV viruses and the grey arrows highlight the ORFs flanking the region of recombination. In the first region, the ORF UL111A has been deleted using bacterial artificial chromosome (BAC) technology. The site of mutation is flanked by the ORFs UL105 and UL112. In the second region, the genomic region encompassing ORFs US2, US3, US6, US7, US8, US9, US10 and US11, denoted by US2-11, is flanked by the ORFs US1 and US12. In the third region the ORFs US15 and US16 are depicted. The direction of the arrows demonstrates the direction of gene transcription.

FIG. 7D: Schematic Diagram of HCMV Merlin-IFNβ UL111A Knock-Out (or HCMV-β ΔUL111A). The HCMV genome is composed of a Unique Long (UL) and Unique Short (US) Region, both flanked by inverted repeat regions (RL and RS). These are depicted by the green and orange boxes, respectively. Each of the genomic regions that underwent subsequent mutation are depicted by red boxes and expanded to highlight the ORFs (shown as arrows) located in the region. The purple arrows depict the ORFs that were deleted during recombination of other modified HCMV viruses and the grey arrows highlight the ORFs flanking the region of recombination. In the first region, the ORF UL111A has been deleted using bacterial artificial chromosome (BAC) technology. The site of mutation is flanked by the ORFs UL105 and UL112. In the second region, the genomic region encompassing ORFs US2, US3, US6, US7, US8, US9, US10 and US11, denoted by US2-11, is flanked by the ORFs US1 and US12. In the third region the ORFs US15 and US16 are depicted. The insertion of the human IFN-β cDNA Cassette, using bacterial artificial chromosome (BAC) technology, between ORFs US15 and US16 is depicted using a blue arrow. The direction of the arrows demonstrates the direction of gene transcription.

FIG. 7E: Schematic Diagram of HCMV Merlin US2-11 Knock-Out (or HCMV ΔUS2-11). The HCMV genome is composed of a Unique Long (UL) and Unique Short (US) Region, both flanked by inverted repeat regions (RL and RS). These are depicted by the green and orange boxes, respectively. Each of the genomic regions that underwent subsequent mutation are depicted by red boxes and expanded to highlight the ORFs (shown as arrows) located in the region. The purple arrows depict the ORFs that were deleted during recombination of other modified HCMV viruses and the grey arrows highlight the ORFs flanking the region of recombination. In the first region, the ORF UL111A is flanked by the ORFs UL105 and UL112. In the second region, the genomic region encompassing ORFs US2, US3, US6, US7, US8, US9, US10 and US11, denoted by US2-11, has been deleted using bacterial artificial chromosome (BAC) technology. The site of mutation is flanked by the ORFs US1 and US12. In the third region the ORFs US15 and US16 are depicted. The direction of the arrows demonstrates the direction of gene transcription.

FIG. 7F: Schematic Diagram of HCMV Merlin-IFNβ US2-11 Knock-Out (or HCMV-β ΔUS2-11). The HCMV genome is composed of a Unique Long (UL) and Unique Short (US) Region, both flanked by inverted repeat regions (RL and RS). These are depicted by the green and orange boxes, respectively. Each of the genomic regions that underwent subsequent mutation are depicted by red boxes and expanded to highlight the ORFs (shown as arrows) located in the region. The purple arrows depict the ORFs that were deleted during recombination of other modified HCMV viruses and the grey arrows highlight the ORFs flanking the region of recombination. In the first region, the ORF UL111A is flanked by the ORFs UL105 and UL112. In the second region, the genomic region encompassing ORFs US2, US3, US6, US7, US8, US9, US10 and US11, denoted by US2-11, has been deleted using bacterial artificial chromosome (BAC) technology. The site of mutation is flanked by the ORFs US1 and US12. In the third region the ORFs US15 and US16 are depicted. The insertion of the human IFN-β cDNA Cassette, using bacterial artificial chromosome (BAC) technology, between ORFs US15 and US16 is depicted using a blue arrow. The direction of the arrows demonstrates the direction of gene transcription.

FIG. 7G: Schematic Diagram of HCMV Merlin UL111A Knock-Out/US2-11 Knock-Out (or HCMV ΔUL111A/ΔUS2-11). The HCMV genome is composed of a Unique Long (UL) and Unique Short (US) Region, both flanked by inverted repeat regions (RL and RS). These are depicted by the green and orange boxes, respectively. Each of the genomic regions that underwent subsequent mutation are depicted by red boxes and expanded to highlight the ORFs (shown as arrows) located in the region. The grey arrows highlight the ORFs flanking the region of recombination. In the first region, the ORF UL111A has been deleted using bacterial artificial chromosome (BAC) technology. The site of mutation is flanked by the ORFs UL105 and UL112. In the second region, the genomic region encompassing ORFs US2, US3, US6, US7, US8, US9, US10 and US11, denoted by US2-11, has been deleted using bacterial artificial chromosome (BAC) technology. The site of mutation is flanked by the ORFs US1 and US12. In the third region the ORFs US15 and US16 are depicted. The direction of the arrows demonstrates the direction of gene transcription.

FIG. 7H: Schematic Diagram of HCMV Merlin-IFNβ UL111A Knock-Out/US2-11 Knock-Out (or HCMV-IFNβ ΔUL111A/ΔUS2-11). The HCMV genome is composed of a Unique Long (UL) and Unique Short (US) Region, both flanked by inverted repeat regions (RL and RS). These are depicted by the green and orange boxes, respectively. Each of the genomic regions that underwent subsequent mutation are depicted by red boxes and expanded to highlight the ORFs (shown as arrows) located in the region. The grey arrows highlight the ORFs flanking the region of recombination. In the first region, the ORF UL111A has been deleted using bacterial artificial chromosome (BAC) technology. The site of mutation is flanked by the ORFs UL105 and UL112. In the second region, the genomic region encompassing ORFs US2, US3, US6, US7, US8, US9, US10 and US11, denoted by US2-11, has been deleted using bacterial artificial chromosome (BAC) technology. The site of mutation is flanked by the ORFs US1 and US12. In the third region the ORFs US15 and US16 are depicted. The insertion of the human IFN-β cDNA Cassette, using bacterial artificial chromosome (BAC) technology, between ORFs US15 and US16 is depicted using a blue arrow. The direction of the arrows demonstrates the direction of gene transcription.

FIG. 8: Fold change in IFNγ+ HCMV VTE-Specific CD8+CD3+ T-cells in response to viral infection. HLA-A1, -A2, -B8 and B57 restricted human fibroblasts (HFs) were either mock infected or infected at a multiplicity of infection (MOI) of 5 with RCMV-IFNβ (human cytomegalovirus engineered to express human interferon beta) or RCMV-Rescuant (RCMV-IFNβ that has had the IFNβ cassette removed) for 16 hours. HLA-A1 restricted cytotoxic CD8+CD3+ T-cells, specific to the HCMV T-cell epitope VTEHDTLLY derived from the human CMV antigen pp50, were subsequently added. After 12 hours T-cell activation was measured by interferon gamma (IFNγ) expression using flow cytometry. The fold change of IFNγ+ CD8+CD3+ T-cells collected from the RCMV-IFNβ and RCMV-Rescuant infections is shown relative to mock infection.

FIG. 9: Schematic Diagram of pCMV6-XL4 IFNβ cDNA Cassette. The IFNβ cDNA cassette is composed of a modified human IFN-β cDNA sequence, represented by the red box, flanked by the CMV Major Immediate early Promoter, represented by the green box, and polyadenylation Poly (A) signal, represented by the yellow box.

FIG. 10: Nucleotide Sequence of pCMV6-XL4 IFNβ cDNA Cassette (SEQ ID No1).

### DETAILED DESCRIPTION OF THE EMBODIMENTS

As described herein, the inventor has found that it is possible to contain CMV to a site of an initial infection, or in other words, to prevent spread of CMV from an initial site of infection to adjacent cells, provided that interferon beta (IFN-β) is also provided at the initial infection site. In the experiments described herein, the inventor has developed nucleic acid constructs in which an IFN-β gene is incorporated into a CMV genome. The expression of the construct enables a production of IFN-β at the site of the CMV genome expression, for example in an initial population of cells infected with the relevant construct. As the latter ostensibly forms an initial infection site, with the constructs of the invention the inventor has developed a technique for targeted delivery or production of IFN-β at an infection site.

As exemplified herein, ‘bystander cells’, or in other words, cells that are located adjacent to cells infected with a nucleic acid or construct of the invention, tend not to become infected with CMV. While not wanting to be bound by hypothesis, it is believed that IFN-β is released from the cells infected with the construct of the invention, for example, when these cells are lysed by CMV particles translated from the construct of the invention. The bystander cells are then contacted with the released IFN-β, one consequence of which is the activation of the IFN-IR signalling pathway and activation of related anti-viral properties in the bystander cells that prevent the bystander cells from being infected with CMV. The finding that activation of the IFN-IR signalling pathway is sufficient to induce an anti-viral response to CMV is surprising given that it had been thought that synergy between IFN-IR and IFN-IIR signalling pathways is required for this response.

Importantly, the inventor has demonstrated that the local production of a Type I interferon (i.e. production at the site of CMV infection) does not prevent the production of CMV proteins at the relevant site. In particular, as exemplified herein, the inventor has detected both the expression of CMV proteins and IFN-β production in cells infected with the construct of the invention. The finding is important because to be useful as a prophylactic or therapeutic, the CMV proteins must be expressed to enable presentation of CMV antigens to T cells (especially CD8⁺ cells) for immune response induction.

Given above, the advantage of the invention is to effectively provide for a local, contained production of CMV proteins for providing systemic immunity to CMV.

‘CMV’ or ‘cytomegalovirus’ is a species of virus that belongs to the viral family known as Herpesviridae or herpesviruses. The form that infects humans is typically abbreviated as HCMV and is alternatively known as Hunan herpesvirus 5 (HHV-5). The invention especially relates to utilising HCMV genes and proteins for application to HCMV infection. Reference to ‘CMV’ in the specification will be understood as including reference to ‘HCMV’.

‘viral particle’ refers to an agent that requires a host cell for replication of the particle.

‘viral life cycle’ refers to the series of steps by which a virus replicates. These may generally include: (i) host cell attachment; (ii) host cell penetration; (iii) unpackaging in the host cell; (iv) viral genome replication; (v) assembly and packaging; (iv) lysis.

‘infectious particle’ refers to an agent that is capable of infecting a host cell, specifically capable of introducing a nucleic acid or construct of the invention into a host cell to enable translation of the respectively encoded gene products by the host cell.

To ‘infect’ generally refers to a process involving one or more steps of the viral life cycle.

Various HCMV genes, especially “UL111a”, “US2”, “US3”, “US4”, “US5”, “US6”, “US7”, “US8”, “US9”, “US10” and “US11” are referred to in preferred embodiments of the invention. As described herein, in certain embodiments, these genes are relevant insofar as encoding immunoregulatory proteins that are deleted in the preferred constructs of the invention. Examples of the sequences of UL111a, US2, US3, US4, US5, US6, US7, US8, US9, US10, US11 are described in Table 1 or Table 2 below. It will be understood that, where referred to in the specification, the relevant gene may have a nucleotide sequence that is at least 60%, preferably 70%, preferably 80%, preferably 85%, preferably 90%, preferably 95%, preferably 98% or 99% or 100% identity to the relevant named accession number in Table 1 or Table 2. It will be understood that the sequences relevant to these genes as referred to by the accession numbers in Table 1 or Table 2 are merely exemplary of the relevant genes. Moreover, where referred to in the specification, UL111a, US2, US3, US4, US5, US6, US7, US8, US9, US10, US11 may have nucleotide sequences that are not the same as those referred to by the accession numbers in Table 1 or Table 2 although they encode UL111a, US2, US3, US4, US5, US6, US7, US8, US9, US10, US11 protein.

Other HCMV genes, especially US15 and US16 are referred to in preferred embodiments of the invention as being non essential genes between which the gene encoding the cytokine of the construct of the invention may be positioned. Examples of the sequences of US15 and US16 are described in Table 1 below. It will be understood that, where referred to in the specification, US5 or US16 may have a nucleotide sequence that is at least 60%, preferably 70%, preferably 80%, preferably 85%, preferably 90%, preferably 95%, preferably 98% or 99% or 100% identity to the relevantly associated accession number in Table 1. Importantly it will be understood that the nucleotide sequences relevant to these genes as referred to by the accession numbers in Table 1 are merely exemplary of the relevant genes. Moreover, where referred to in the specification, US15 and US16 may have nucleotide sequences that are not the same as those referred to by the accession numbers in Table 1 although they encode US15 or US16 protein.

TABLE 1
Relative N Positions of HCMV Genes on HCMV Merlin

| | Nucleotide Position of Gene | Gene |
|---|---|---|
| Gene | Encoded by HCMV Merlin | Identity Number |
| UL111a | 161003 . . . 161692 | 3077506 |
| US2 | 199331 . . . 199930 | 3077542 |
| US3 | 200345 . . . 200905 | 3077532 |
| US6 | 201615 . . . 202163 | 3077555 |
| US7 | 202589 . . . 203266 | 3077535 |
| US8 | 203469 . . . 204152 | 3077426 |
| US9 | 204167 . . . 204910 | 3077455 |
| US10 | 205296 . . . 205853 | 3077551 |
| US11 | 205929 . . . 206576 | 3077490 |
| US15 | 209518 . . . 210306 | 3077565 |
| US16 | 210366 . . . 211295 | 3077558 |

Regarding Table 1, the nucleotide positions, represented as nucleotide numbers, of HCMV ORFS encoding genes UL111A, US2, US3, US6, US7, S8, US9, US10, US11, US15 and US16 as encoded by HCMV Merlin (Accession Number: NC_006273.2) is shown in the middle column. The gene identity number is shown in the right hand column.

TABLE 2
Relative N Positions of HCMV Genes
in strain AD169 (Acc. No: X17403.1)

| | Nucleotide Position of Gene | Protein |
|---|---|---|
| Gene | Encoded by HCMV AD169 | Identity Number |
| US4 | 194832 . . . 195191 | CAA35271.1 |
| US5 | 195230 . . . 195610 | P16840 |

Regarding Table 2, the nucleotide positions, represented as nucleotide numbers, of HCMV ORFS encoding genes US4 and US5 as encoded by HCMV strain AD169 (Acc. No: X17403.1) is shown in the middle column. The protein identity number is shown in the right hand column.

‘comprise’ and variations of the term, such as ‘comprising’, ‘comprises’ and ‘comprised’, are not intended to exclude further additives, components, integers or steps, except where the context requires otherwise.

In one embodiment there is provided a nucleic acid or construct including:
a first region encoding:
   a cytomegalovirus (CMV); or
   a viral particle including a CMV protein;
a second region encoding a cytokine for preventing a CMV or viral particle translated from the first region from infecting a cell.

In accordance with the invention, the nucleic acid is translated into the respectively encoded gene products in the host cell. The gene products may result in the formation of a complete CMV (i.e. a CMV having all of the protein domains and functionalities found in wild type CMV) or a partial CMV (i.e. a CMV lacking certain protein domains).

In one embodiment, a cell infected with the nucleic acid of this embodiment may produce CMV that more or less has the same capacity for infection as a wild type CMV. For example, the cell may be capable of producing a virus that is capable of completing all steps of the CMV viral lifecycle. Alternatively, a cell infected with the nucleic acid of this embodiment may simply produce an assortment of CMV peptides encoded by the first region which is then either unable to assemble into a CMV particle, or otherwise, if assembled, is unable to lyse the host cell, to form infective virus.

In a preferred embodiment, the nucleic acid of the invention is capable of producing a CMV that has the same capacity for infection or viral replication as a wild type CMV, as such a virus is expected to contain the full spectrum of viral epitopes found in nature, and therefore more likely to generate an effective polyclonal response when challenged with wild type CMV. Typically a virus or viral particle encoded by the nucleic acid or construct of the invention has the same capacity (i.e. 100% capacity) for viral replication as compared to a wild type CMV, especially a CMV that does not contain a second region encoding a cytokine for preventing a CMV or viral particle translated from the first region from infecting a cell, and preferably a capacity greater than 50%, 75%, 80%, 85%, 90%, 95%, 99%.

In one embodiment, the nucleic acid of the invention may have a loss of function mutation in, or deletion of, a gene encoding a ‘non-essential’ protein. These genes are generally not required for replication in vivo. Examples of these genes are disclosed in Yu et al. 2003 PNAS USA 100:12396, and include US1, UL23, UL116, US10, US31, UL2, UL24, UL118, US11, US32, UL3, UL25, UL119, US12, US33, UL4, UL27, UL120, US13, US34, UL5, UL31, UL121, US14, RL1, UL6, UL33, UL124, US15, RL2, UL7, UL35, UL128, US16, RL4, UL8, UL36, UL130, US17, RL6, UL9, UL37.3, UL132, US18, RL9, UL10, UL40, UL146, US19, RL10, UL11, UL41, UL147, US20, RL11, UL13, UL42, US1, US21, RL12, UL14, UL43, US2, US22, RL13, UL15, UL45, US3, US24, UL16, UL65, US5, US25, UL17, UL78, US6, US27, UL18, UL83, US7, US28, UL19, UL88, US8, US29, UL20, UL111a, US9, US30. Generally, a virus that has absence of a functional non essential gene has the same capacity for viral replication as one that contains a functional non essential gene, although in some circumstances the capacity for replication may be less where the gene, while not critical for replication, otherwise has a role in enhancing replication. In these circumstances, deletion of a gene encoding a ‘non essential’ protein may diminish, but not ablate replication capacity.

Preferably the nucleic acid of the invention includes genes encoding ‘essential proteins’. These are proteins that are need for viral replication in vivo. They include IE1/2, UL37xl, UL44, UL51, UL52, UL53, UL56, UL77, UL79, UL84, UL87 and UL105.

In another embodiment, the gene products may also result in the formation of a viral particle, for example a virus that expresses 1, 2, 10, 100 or 200 or more CMV epitopes. In this embodiment, the CMV epitopes may be carried by a viral vector which may also be encoded by the first region. The infectiousness of the viral particle—i.e. the capacity of the particle to complete the steps of a viral life cycle—may arise from the viral vector, rather than from the encoded CMV epitopes. One example of a viral vector is a virus related to herpes, or to vaccinia. In this example, the first region of the nucleic acid or construct of the invention has the sequence of vaccinia and has CMV genes located in the vaccinia sequence. The purpose of the vaccinia sequence is to enable formation of a viral particle. The purpose of the CMV genes is to encode immunogenic peptides for invoking immunity to CMV. Thus in one embodiment there is provided a nucleic acid or construct including:
a first region including a viral genome for forming viral particle, said genome being other than a CMV genome, preferably a vaccinia genome, and having one or more genes encoding immunogenic CMV-derived peptides contained within;
a second region encoding a cytokine for preventing a viral particle translated from the first region from infecting a cell.

Preferably the first region contains CMV genes encoding the immunodominant CMV epitopes. These epitopes are those that activate common T cell subsets in the majority of the infected population. Examples of immunodominant CMV epitopes or genes encoding same include those useful for stimulating a predominant CD4+ response, especially UL55 (gB), UL83 (pp65), UL86, UL99 (pp28), UL122 (IE2), UL36, UL48, UL32, and UL113; and those useful for stimulating a predominant CD8+ response, especially UL48, UL83, UL123, UL122, US32, UL28, US29, US3, UL32, UL55, UL94, and UL69.

According to the invention, the cytokine produced by the second region is for preventing a CMV or viral particle translated from the first region from infecting a cell. In more detail, upon translation of the nucleic acid in the host cell, a cytokine is produced from the second region. That cytokine may be released from the host cell for contact with bystander cells. When contacted with the cytokine, the interferon stimulated genes of the bystander cells may be activated to provide the bystander cells with an anti-viral phenotype. This may prevent the CMV or viral particle that is translated from, or produced from the first region of nucleic acid in the host cell, from infecting bystander cells. Thus the cytokine may prevent one or more of the following steps from completion in respect of CMV or viral particle produced from an infected cell: (i) bystander cell attachment; (ii) bystander cell penetration; (iii) unpackaging in the bystander cell; (iv) viral genome replication in the bystander cell; (v) assembly and packaging in the bystander cell; (iv) lysis of the bystander cell.

In certain embodiments, the production of the cytokine in the host cell initially infected with the nucleic acid of the invention may prevent the CMV or viral particle translated from the nucleic acid from completing one or more steps of the viral lifecycle in the host cell. Put in other words, the cytokine may activate response genes in the host cell (in contrast to the above described embodiment where response genes are activated in a bystander cell), leading to prevention of one or more of the following steps in the host cell: (i) viral genome replication; (ii) viral assembly and packaging; (iii) lysis.

The location of the first region relative to the second region in the nucleic acid or construct of the invention is unimportant, provided that both the first and second regions are translated into the respectively encoded gene products when the nucleic acid of the invention is introduced into a cell. It will be understood that the first region may be located 5′ to the second region, or the second region may be located 5′ to the first region.

In one embodiment, the second region is located within the first region.

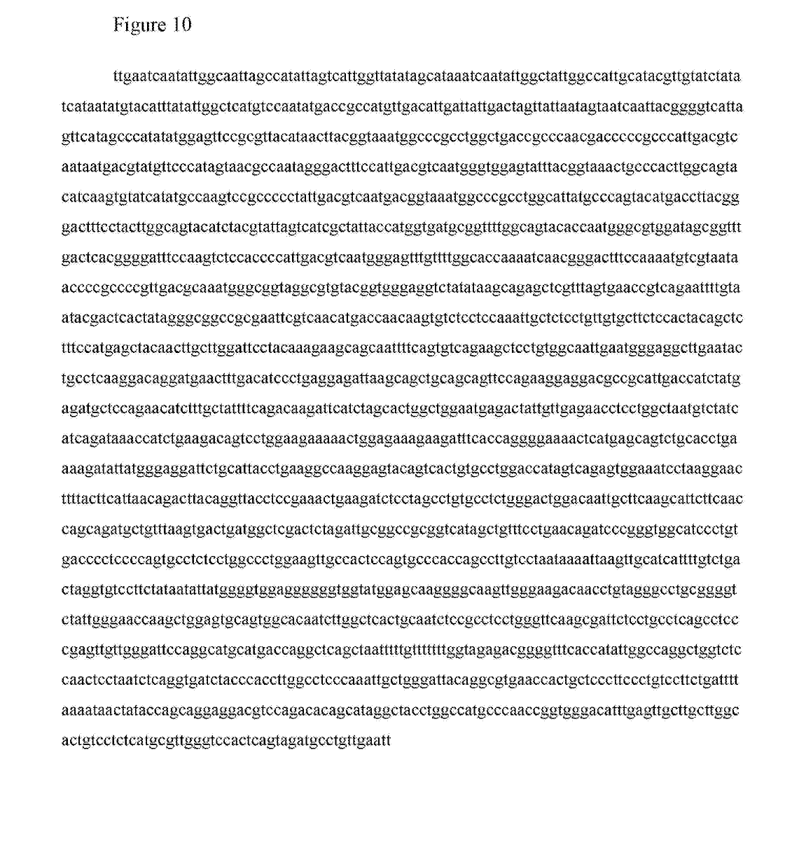

In a particularly preferred embodiment, the second region is located between US15 and US16 of the US region of the CMV genome. One advantage of this location is that there is a large segment between US15 and US16 in which a heterologous gene may be placed. Further, both US15 and US16 are non essential genes for replication. The nucleotide sequence of a fragment of a construct in which an IFN-β gene is located between US15 and US16 is show in FIG. 10 (SEQ ID No:1).

Other locations within the first region are possible and these locations may depend on whether the CMV or viral particle has some or all of the genes typically found in the genome of a CMV strain. In one embodiment, the second region is located between non essential genes of CMV, or is located so as to completely or partially excise non essential genes of CMV. Preferably the heterologous gene, especially the cytokine encoding gene of the second region, is inserted into a non coding region between non essential genes.

In one embodiment, the second region is operably linked to a promoter or regulatory element for enabling transcription or expression of the second region independently of transcription of the first region. A variety of promoters may be used to regulate expression of the second region. It is not necessary that the relevant promoter be derived from CMV.

Preferably the promoter enables transcription of the second region before transcription of the first region. In particular, it is believed that it is important that IFN-β is provided to the bystander cells so as to appropriately condition the anti-viral properties of these cells before they are exposed to CMV. In one embodiment the promoter is a CMV promoter, preferably a MEI or IE promoter. Other promoters include: SV40 promoter, HSV-1 ICPO or TK promoter, actin promoter, EF 1-a promoter, Ubc promoter and PGK promoter.

The promoter for the second region may also control the transcription of one or more genes of the first region. In this embodiment, the first and second regions may be arranged so as to enable a single promoter to control expression of both regions.

In one embodiment, the promoter controlling transcription or expression of the second region is an inducible promoter. This enables the targeted expression of the IFN-β from the second region in a dose dependent and time controlled manner.

In one embodiment, the first region includes an attenuation of a CMV gene that prevents formation of a CMV or viral particle that is capable of completing the steps of a viral life cycle. For example, the attenuation may be in a gene involved in (i) cell attachment; (ii) cell penetration; (iii) unpackaging in the cell; (iv) viral genome replication; (v) assembly and packaging; (iv) lysis. An attenuation may include the complete or partial deletion or excision of a gene or fragment thereof, or an insertion of sequence into a gene, the result of which is to cause a loss of function.

In another embodiment the first region includes an attenuation of a CMV gene that regulates expression of host MHC Class I or II genes. The impact of the attenuation is generally to increase the expression, or to restore the expression of Class I and/or Class II molecules to the cell surface of an infected cell, thereby increasing antigen presentation to T cells. Importantly, CMV is known to contain a number of genes that impact on the immune surveillance of infected cells, potentially decreasing the immunogenicity of these cells. Viral IL-10 is one example.

In the above described embodiments, the attenuation may delete the function of the relevant gene, or modify the function so as to inhibit or prevent the completion of the one or more of the stages of the viral life cycle.

In one embodiment, the nucleic acid of the invention contains an attenuation in one or more of the following genes: UL111a (viral IL-10), US2, US3, US4, US5, US6, US7, US8, US9, US10 and US11 genes. In one embodiment the nucleic acid of the invention does not include one or more of the following CMV genes: UL11a (viral IL-10), US2, US3, US4, US5, US6, US7, US8, US9, US10 and US11 genes.

In certain embodiments the first region may include a mutation for modifying the immunogenicity of the CMV or viral particle encoded by the first region.

In other embodiments, the nucleic acid may include one or more genes encoding a non CMV epitope. For example, a CMV encoded by a nucleic acid of the invention may further include an immunogenic non CMV epitope (for example from HSV, vaccinia, HIV) for effectively potentiating an immune response to CMV. In this context the non CMV epitope could act as an adjuvant.

Typically, the cytokine encoded by the second region is one capable of activating the anti-viral properties of the host cell or bystander cell. Examples of these properties include those that directly or indirectly target one or more stages of the viral lifecycle. In one particularly preferred embodiment, the cytokine activates the IFN-IR pathway. In another preferred embodiment, the cytokine activates expression of MHC Class I or II molecules on the host cell or bystander cell surface.

In one embodiment, the second region encodes a cytokine in the form of an interferon Type I (IFNI), and especially IFN-β.

In one embodiment, in addition to encoding IFN-β, the nucleic acid encodes a further cytokine for preventing a CMV or viral particle translated from the first region from infecting a cell. One example is IFN-γ.

In one embodiment, the only cytokine encoding gene contained in second region is the IFN-β gene In this embodiment, the nucleic acid or construct of the invention does not contain a gene encoding a IFN-α or IFN-γ.

In certain embodiments, the first region is linked to the second region thereby encoding a fusion protein in the form of a CMV peptide or viral particle peptide encoded by the first region that is linked to the cytokine encoded by the second region.

In one embodiment, there is provided: a nucleic acid or construct including:
a first region encoding CMV; and
a second region encoding IFN-β,
   wherein the first region contains an attenuation of UL111a and US2.

In one embodiment, there is provided:

a nucleic acid or construct including:
a first region encoding CMV; and
a second region encoding IFN-β,
   wherein the first region contains an attenuation of UL111a and US3.

In one embodiment, there is provided:

a nucleic acid or construct including:
a first region encoding CMV; and
a second region encoding IFN-β,
   wherein the first region contains an attenuation of UL111a and US4.

In one embodiment, there is provided:

a nucleic acid or construct including:
a first region encoding CMV; and
a second region encoding IFN-β,
   wherein the first region contains an attenuation of UL111a and US5.

In one embodiment, there is provided:

a nucleic acid or construct including:
a first region encoding CMV; and
a second region encoding IFN-β,
   wherein the first region contains an attenuation of UL111a and US6.

In one embodiment, there is provided:

a nucleic acid or construct including:
a first region encoding CMV; and
a second region encoding IFN-β,
   wherein the first region contains an attenuation of UL111a and US7.

In one embodiment, there is provided:

a nucleic acid or construct including:
a first region encoding CMV; and
a second region encoding IFN-β,
   wherein the first region contains an attenuation of UL111a and US8.

In one embodiment, there is provided:

a nucleic acid or construct including:
a first region encoding CMV; and
a second region encoding IFN-β,
   wherein the first region contains an attenuation of UL111a and US9.

In one embodiment, there is provided:

a nucleic acid or construct including:
a first region encoding CMV; and
a second region encoding IFN-β,
   wherein the first region contains an attenuation of UL111a and US10.

In one embodiment, there is provided:

a nucleic acid or construct including:
a first region encoding CMV; and
a second region encoding IFN-β,
   wherein the first region contains an attenuation of UL111a and US11.

In one embodiment, there is provided:

a nucleic acid or construct including:
a first region encoding CMV; and
a second region encoding IFN-β,
   wherein the first region contains an attenuation of UL111a and US2, US3, US4, US5, US6, US7, US8, US9, US10, and US11.

One skilled in the art can use viral replication assays to confirm the activity of a nucleic acid or construct of the invention, or virus or particle encoded by same.

In one embodiment, viral titers are determined by a 50% Tissue Culture Infective Dose (TCID50) assay. Briefly, this dilution assay quantifies the amount of virus required to kill 50% of infected hosts. Host cells are plated and serial dilutions of the virus are added. After incubation, the percentage of cell death (i.e. infected cells) is observed and recorded for each virus dilution. Results are used to mathematically calculate the TCID50.

In another embodiment, the viral titers are determined using a plaque assay. Viral plaque assays determine the number of plaque forming units (pfu) in a virus sample. Briefly, a confluent monolayer of host cells is infected with the virus of the invention at varying dilutions and covered with a semi-solid medium, such as agar or carboxymethyl cellulose, to prevent the virus infection from spreading indiscriminately. A viral plaque is formed when a virus infects a cell within the fixed cell monolayer. The virus infected cell will lyse and spread the infection to adjacent cells where the infection-to-lysis cycle is repeated. The infected cell area will create a plaque (an area of infection surrounded by uninfected cells) which can be seen visually or with an optical microscope. Plaques are counted and the results, in combination with the dilution factor used to prepare the plate, are used to calculate the number of plaque forming units per sample unit volume (pfu/mL). The pfu/mL result represents the number of infective particles within the sample and is based on the assumption that each plaque formed is representative of one infective virus particle.

In another embodiment, a hu-SCID mouse model is used to evaluate the ability of an virus of the invention to replicate in vivo. Briefly, pieces of human fetal tissues (such as thymus and liver) are surgically implanted in kidney capsules of SCID mice. The virus is inoculated 2-3 months later when the human tissues are vascularized. Viral titers are assessed 3-4 weeks after inoculation in plaque assays. In addition, humanised mice can also be reconstituted with human immune cells from bone marrow or peripheral blood to assess immune response to infection, and challenge.

In one embodiment, a composition comprising the CMV or viral particle of the invention has a viral titer of at least 10⁵ pfu/ml, more preferably at least 10⁷ pfu/ml.

The nucleic acids of the invention may be used for production of virus and infective particles that can be used for inducing or potentiating an immune response to CMV in an individual. The nucleic acids may be administered directly to an individual for inducing or potentiating an immune response to CMV.

Diseases or conditions that may be treated, prevented, or exhibit an alleviation of symptoms according to the present invention include any disease or condition that involves the acute or latent infection by cytomegalovirus. It will be appreciated by those skilled in the art that reference herein to treatment extends to prophylaxis as well as the treatment of established infections or symptoms.

The vaccine may be used to immunize any individual. By “individual” is meant any animal, for example, a mammal, or, for example, a human, including, for example, patients in need of treatment. A nucleic acid of the invention in the form of a HCMV vaccine could, depending on its protective ability, be sold and administered by physicians or clinics to either specific at risk populations or possibly to all children as part of childhood immunization. By “at risk” population is meant any individual at risk for diseases caused by HCMV, or that may carry HCMV infection to others including, for example, women before their child-bearing years, children, children under age one, day care providers, and transplant recipients or transplant donors. By treating transplant donors, for example, before the transplant is donated, infection of the recipient may be avoided. Transplants may include, for example, hematopoietic stem cell and solid organ transplants.

In one embodiment, the immunisation protocol follows a priming arm followed by a boosting arm. The nucleic acid of the invention may be provided for use in the priming arm in the form of a DNA vaccine, or in the form of infectious virus. Likewise, the boost arm may be provided by utilization the nucleic acid of the invention in the form of a DNA vaccine, or in the form of infectious virus. It is possible to prime with the nucleic acid of the invention as a DNA vaccine, and then boost with an infectious virus including the nucleic acid molecule of the invention, or vice versa.

Generally the purpose of the immunisation procedure is to raise an effective CD8+ cytotoxic T cell response. This may be the focus of the priming arm, whereas a boosting arm with, for example an infectious particle including the nucleic acid of the invention may additionally elicit virus neutralizing antibodies that help limit the dissemination of virus.

The direct inoculation of plasmid or ‘naked’ DNA into animal tissues has become a widely used approach to vaccination as it overcomes many of the dangers and limitations associated with traditional immunization methods. DNA immunization has been shown to generate protective humoral and cell-mediated responses in a variety of infectious disease models, but its ability to present antigen-derived peptides on MHC class I complexes and generate anti-viral CD8+ T lymphocytes is the key correlate for protection against CMV disease. Several methods for delivering pDNA have been developed to effectively generate immune responses, including biolistic (gene gun) delivery using microprojectiles, intramuscular (i.m.) and intradermal (i.d.) injections (administered by needle or Bioject needleless jet injection), and mucosal delivery.

The DNA vaccine including the nucleic acid of the invention may be administered using any appropriate method, including, for example using a replicating or a non-replicating viral vector, such as, for example, an adenovirus or vaccinia virus vector, a purified plasmid vector, or other form of DNA vaccine known to those of ordinary skill in the art. Exemplary methods are discussed in Vaccine. Volume 30, Issue 49, 19 Nov. 2012, Pages 6980-6990. The next generation recombinant human cytomegalovirus vaccine candidates-Beyond gB. Anders E. Lilja, Peter W. Mason.

An immune response elicited by the CMV or viral particle of the invention can be assessed using methods known in the art. In one embodiment, immune sera from individuals administered with the CMV or viral particle of the invention can be assayed for neutralizing capacity, including but not limited to, blockage of pathogen attachment or entry to a host cell. In other embodiments, T cells from individuals administered with the CMV or viral particle of the invention can be assayed for cytokine producing capacity including, but not limited to, interferon gamma, in the presence of an antigen of interest. Animal challenge models can also be used to determine an immunologically effective amount of immunogen.

Neutralization refers to pathogen specific antibodies capable of interrupting pathogen entry and/or replication in cultures. The common assay for measuring neutralizing activities for viruses is viral plaque reduction assay. Neutralizing activity for pathogens that do not enter cells can be assays by reduction in pathogen replication rates. NT50 titers are defined as reciprocal serum dilutions to block 50% of input pathogen in pathogen neutralization assays. NT50 titers are obtained from nonlinear logistic four-parameter curve fitting.

The present invention encompasses methods of making the CMV or viral particle of the invention. In some embodiments of the invention, the CMV or viral particle of the invention are propagated on epithelial cells, preferably human epithelial cells, and more preferably human retinal pigmented epithelial cells or fibroblasts, more preferable human fibroblasts. Examples include ARPE-19 cells deposited with the American Type Culture Collection (ATCC) as Accession No. CRL-2302 MRC-5 cells deposited with the ATCC as Accession No. CCL-171.

In some embodiments, the cells used to propagate the CMV or viral particle of the invention are grown on microcarriers. A microcarrier is a support matrix allowing for the growth of adherent cells in spinner flasks or bioreactors (such as rotating wall microgravity bioreactors and fluidized bed bioreactors). Microcarriers are typically 125-250 μM spheres with a density that allows them to be maintained in suspension with gentle stirring. Microcarriers can be made from a number of different materials including, but not limited to, DEAE-dextran, glass, polystyrene plastic, acrylamide, and collagen. The microcarriers can have different surface chemistries including, but not limited to, extracellular matrix proteins, recombinant proteins, peptides and charged molecules. Other high density cell culture systems, such as Corning HyperFlask® and HyperStack@systems can also be used.

The cell-free tissue culture media can be collected and CMV or viral particle of the invention can be purified from it. CMV viral particles are about 200 nm in diameter and can be separated from other proteins present in the harvested media using techniques known in the art including, but not limited to ultracentrifugation through a density gradient or a 20% Sorbitol cushion. The protein mass of the vaccines can be determined by Bradford assay.

Further aspects of the present invention and further embodiments of the aspects described in the preceding paragraphs will become apparent from the following description, given by way of example and with reference to the accompanying drawings.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1

### Generation of Recombinant CMV-IFN-β

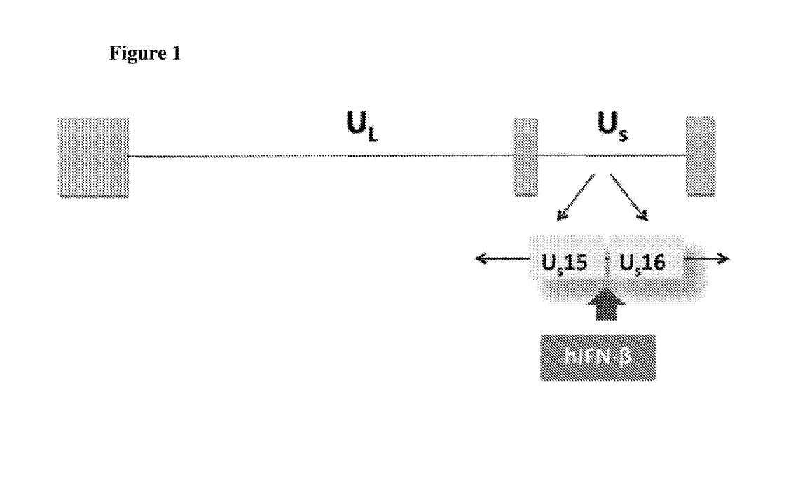

Using bacterial artificial chromosome (BAC) technology, human IFN-β cDNA was inserted in between the non-essential viral genes U_{S}15 and U_{S}16 of the GFP-tagged HCMV clinical isolate Merlin³ (FIG. 1).

### Example 2

### Production of IFNb by Cells Infected with CMV-IFN-β

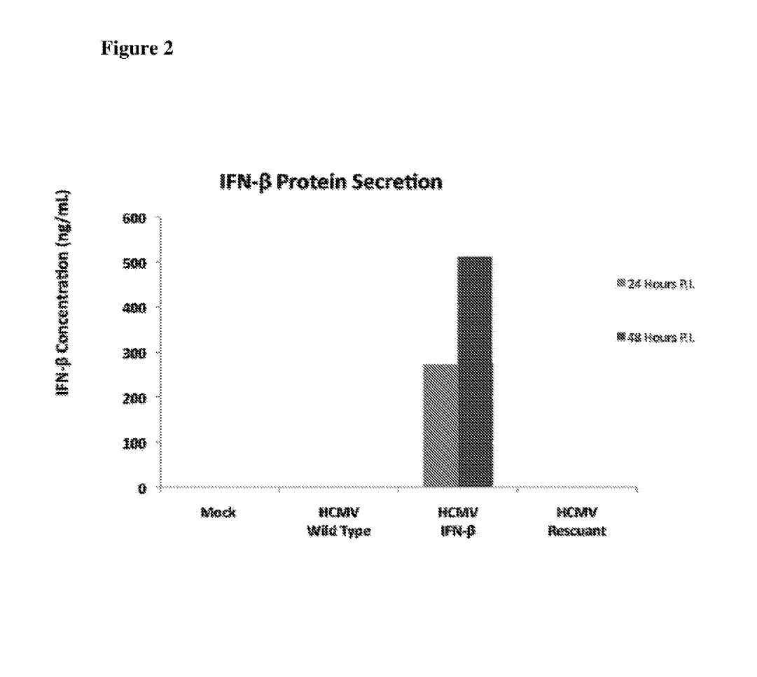

In order to determine whether Merlin HCMV IFN-β increases the expression of human IFN-β by infected HFFs, a human IFN-β ELISA was performed on supernatants generated from an infection of HFFs with HCMV Wild Type, HCMV IFN-β or HCMV Rescuant, in which the human IFN-β cDNA was removed. Analysis revealed that HFFs infected with HCMV IFN-β produced a higher concentration of human IFN-t compared with cells infected with HCMV Wild Type or HCMV Rescuant (FIG. 2).

### Example 3

### Growth Attenuation in HFF Cells Infected with CMV-IFN-β

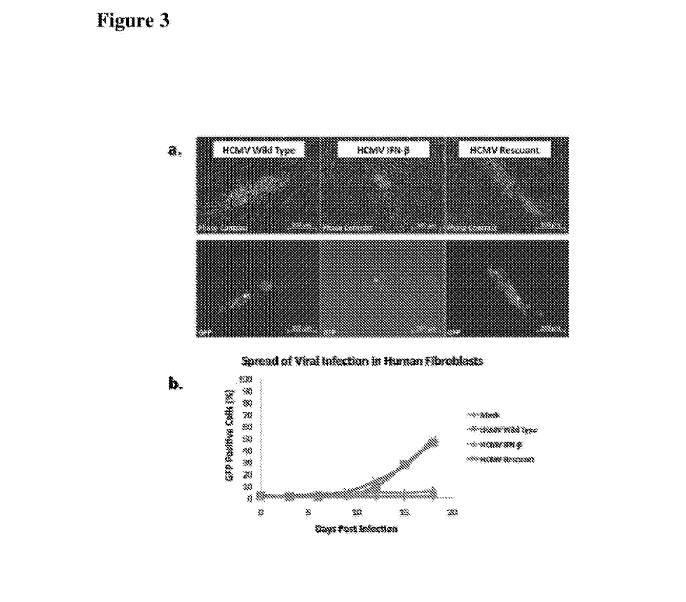

HFFs were infected with HCMV Wild Type, HCMV IFN-β or HCMV Rescuant at a low MOI in order to assess the growth characteristics of HCMV IFN-β virus. Analysis revealed that HCMV IFN-β was severely growth attenuated in HFFs (FIG. 3).

### Example 4

### Growth Attenuation Arises from IFN-β

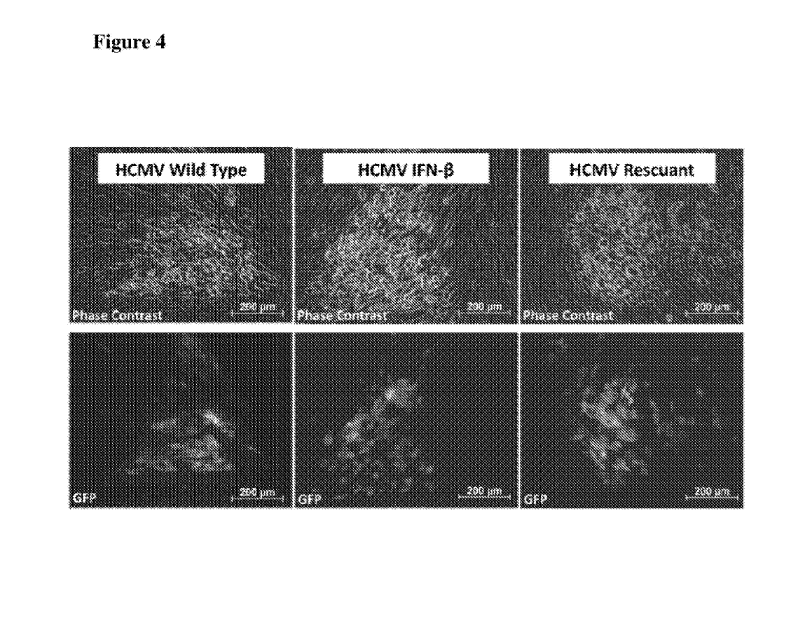

In order to assess whether the production of human IFN-β by HCMV IFN-D was responsible for it's attenuated growth in HFFs, HFFs were generated that were resistant to the effects of interferons. Using a lentiviral transduction system, HFFs were engineered to express the V protein of parainfluenza virus 5 (PIV-5) that targets STAT1 (essential for the downstream signalling of interferons) for proteosome-mediated degradation. Unlike the severe viral growth attenuation observed in HFFs, HCMV IFN-β demonstrated normal growth characteristics, when compared to HCMV Wild Type or HCMV Rescuant, in the interferon resistant HFFs (FIG. 4).

### Example 5

### IFNβ Induces ISGs in CMV-IFN-β Infected Cells

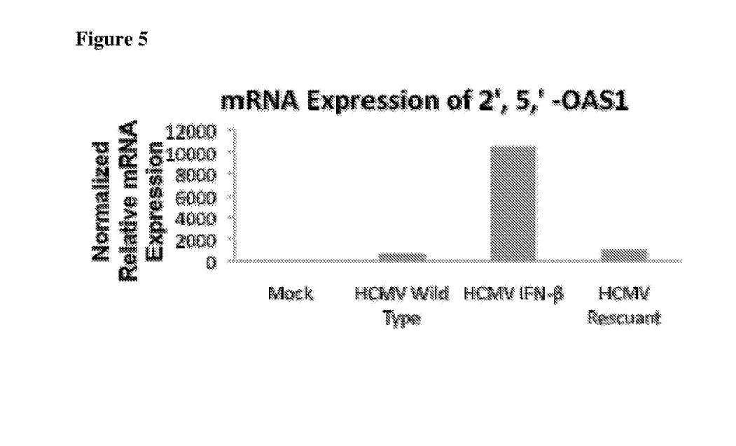

The increased production of human IFN-β by HCMV IFN-β is predicted to lead to an increase in the induction of ISGs which act to block viral replication and therefore limit viral spread. In order to assess whether the attenuated viral growth of HCMV IFN-1 in HFFs may be attributable to increased induction of ISGs the mRNA expression of 2′,5′, OAS1 (an ISG known to block viral replication) was assessed. Analysis revealed that upon infection of HFFs, HCMV IFN-β increased the expression of ISGs, such as 2′,5′,-OAS1 (FIG. 5).

### Example 6

### IFN-β Protects Bystander Cells from Infection

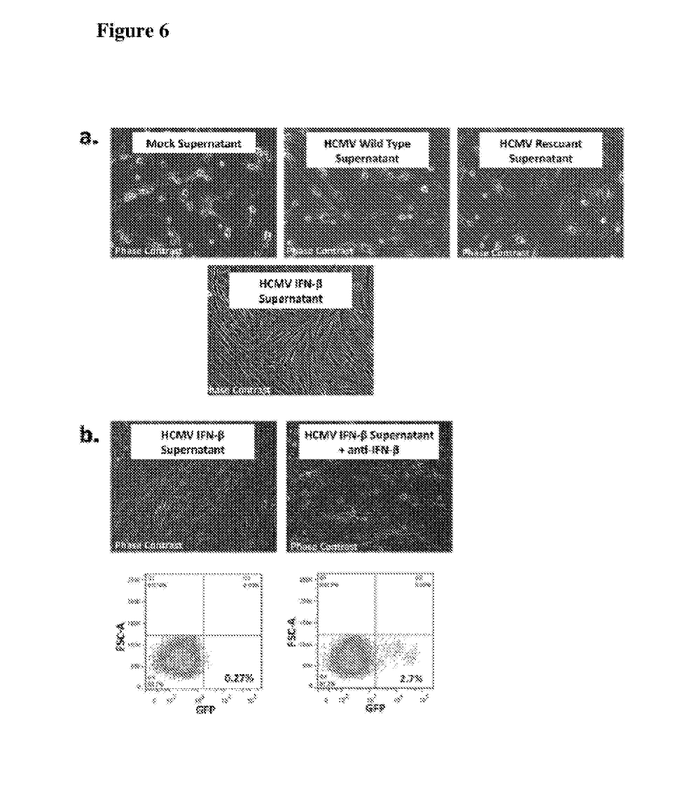

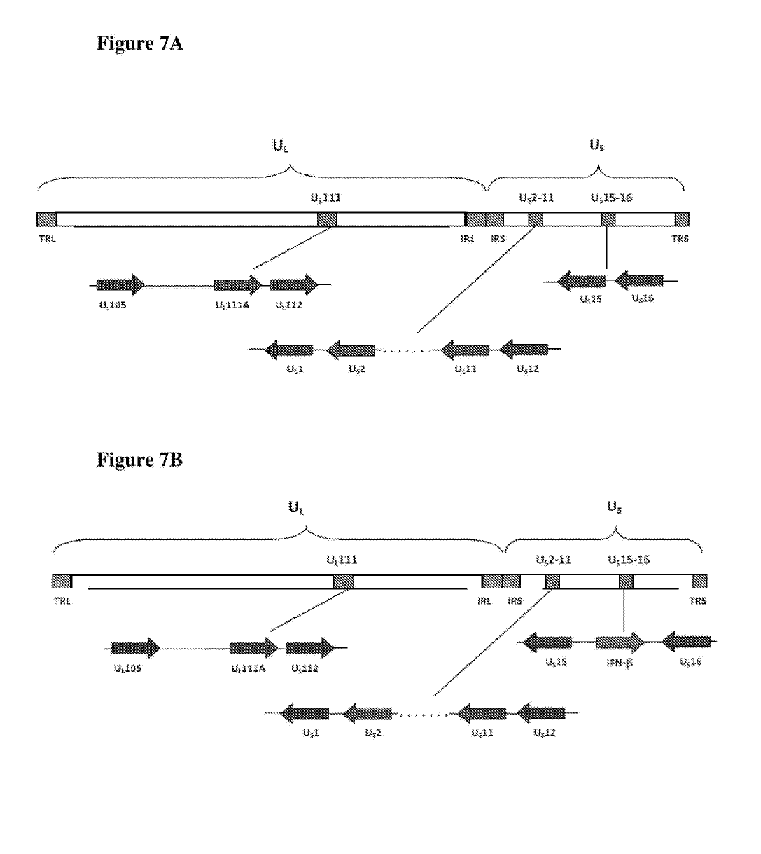

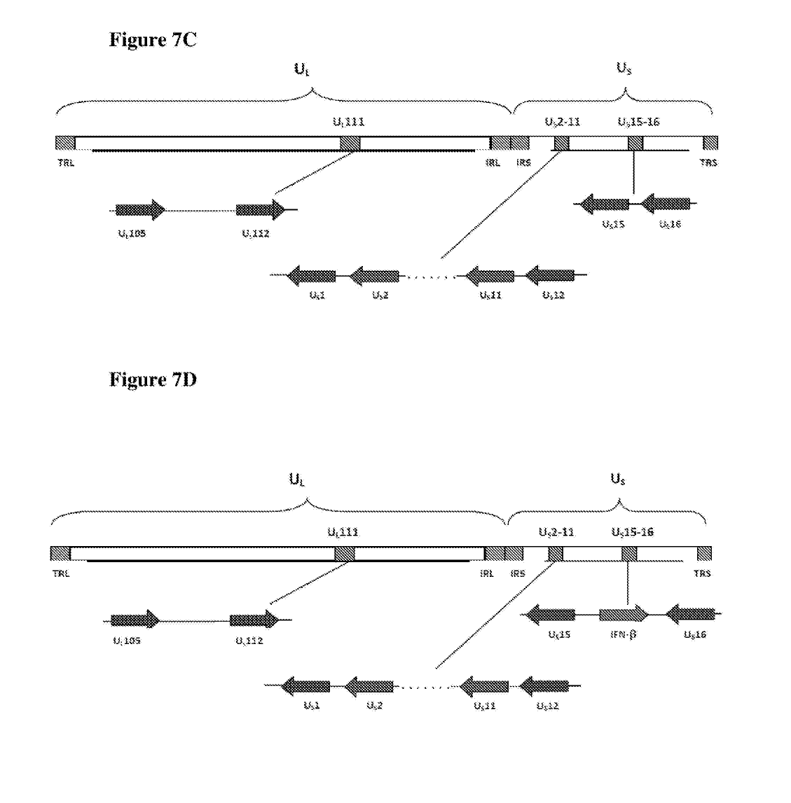

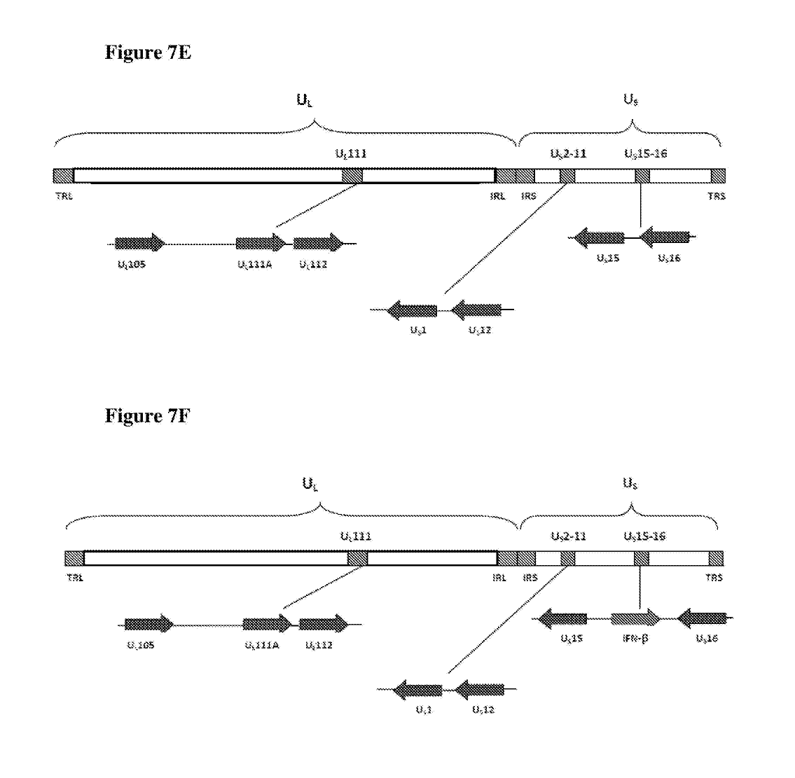

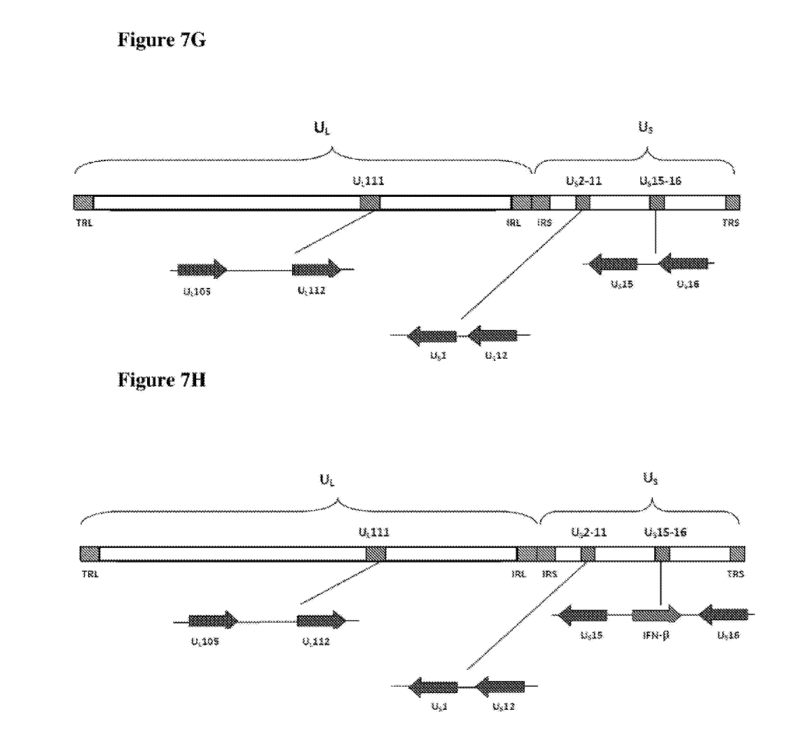

In order to confirm the antiviral properties of the human IFN-β produced by HCMV IFN-β on uninfected bystander cells, virus-free supernatant was collected from HFFs either mock infected, or infected with HCMV Wild Type, HCMV IFN-β or HCMV Rescuant viruses 40 hours post infection. The supernatant was incubated with permissive HFFs 24 hours prior to exposing these pre-treated cells to HCMV Wild-Type. It was observed that cells pre-treated with the supernatant from HCMV IFN-(3 were protected from the subsequent viral infection (FIG. 6).

### Example 7

### Memory T Cells from CMV Infected Individuals are Activated by CMV-IFN-β Infected Cells

The following experiments may be conducted to determine activation of memory T cells from CMV infected individuals by CMV-IFN-β infected cells:

(i) culture memory cells in presence of antigen presenting cells, CMV derived peptide and supernatant obtained from CMV/IFNβ transfection; this demonstrates the effect of supernatant on response of memory cells.

(ii) culture memory cells with CMV/IFNβ transfectants; this demonstrates presentation of antigen to memory cells by transfectants.

(iii) culture memory cells with antigen presenting cells, and supernatant from CMV/IFNβ transfection; this demonstrates presentation by antigen presenting cells of antigen in supernatant to memory cells.

### Example 8

### Mice Exposed to CMV-IFNβ are Protected from Infection with CMV

In humanised mice: Infect humanised mice with human CMV vaccine candidate. Monitor induction of CMV-specific human T cells. Challenge mice by infection with wild-type HCMV. Monitor the extent of infection (viral load, number of cells infected, proliferation of CMV specific human T cells). Control would be vaccination with PBS (or control viruses such as the rescuant) followed by the same challenge with wild type CMV. This analysis could possibly be included to determine whether the vaccine could prevent reactivation from latency (see references below using humanised mice to study human CMV latency/reactivation)

In murine CMV: Insert IFN-beta into murine CMV. Vaccine with murine CMV-IFNbeta virus. Monitor formation of MCMV specific T cells. Challenge mice with wild type MCMV and monitor clearance of virus etc (as in the above example).

### Example 9

### CMV Disease is Cleared in Mice Treated with CMV-IFNβ

For disease, infect with wild type CMV and then inoculate with IFNbeta expressing CMV (either in humanised mice, using human CMV viruses) or in normal mice, using murine CMV as the inoculating virus, and murine CMV expressing IFN-beta, as the virus being tested for impacts on virus clearance. Measure infectious virus clearance in various tissues (eg lung, salivary gland, liver).

Examples of humanised mouse models for use in Examples 8 and 9 are discussed in Hepatol Res. 2013 June, 43(6):679-84. doi: 10.1111/j.1872-034X.2012.01116.x. Epub 2013 Feb. 26. Human cytomegalovirus infection in humanized liver chimeric mice. Kawahara T, Lisboa L F, Cader S. Douglas D N, Nourbakhsh M, Pu C H, Lewis J T, Churchill T A, Humar A, Kneteman N M; Cell Host Microbe. 2010 Sep. 16; 8(3):284-91. doi: 10.1016/j.chom.2010.08.001. Granulocyte-colony stimulating factor reactivates human cytomegalovirus in a latently infected humanized mouse model. Smith M S, Goldman D C, Bailey A S, Pfaffle D L, Kreklywich C N, Spencer D B, Othieno F A, Streblow D N, Garcia J V, Fleming W H, Nelson J A. And PLoS Pathog. 2011 December; 7(12):e1002444. doi: 10.1371/journal.ppat.1002444. Epub 2011 Dec. 29. A novel human cytomegalovirus locus modulates cell type-specific outcomes of infection. Umashankar M, Petrucelli A, Cicchini L, Caposio P, Kreklywich C N, Rak M, Bughio F, Goldman D C, Hamlin K L, Nelson J A, Fleming W H, Streblow D N, Goodrum F.

### Example 10

### Immunogenicity of CMV-IFNβ

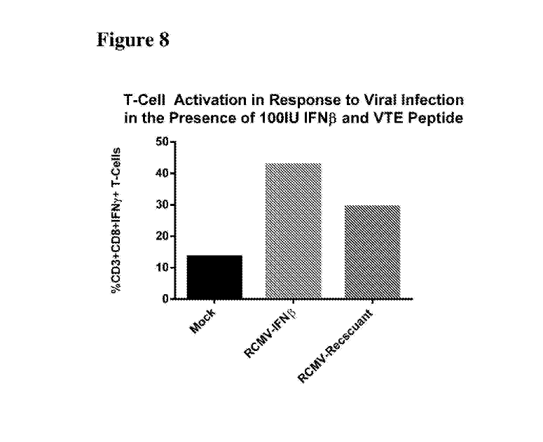

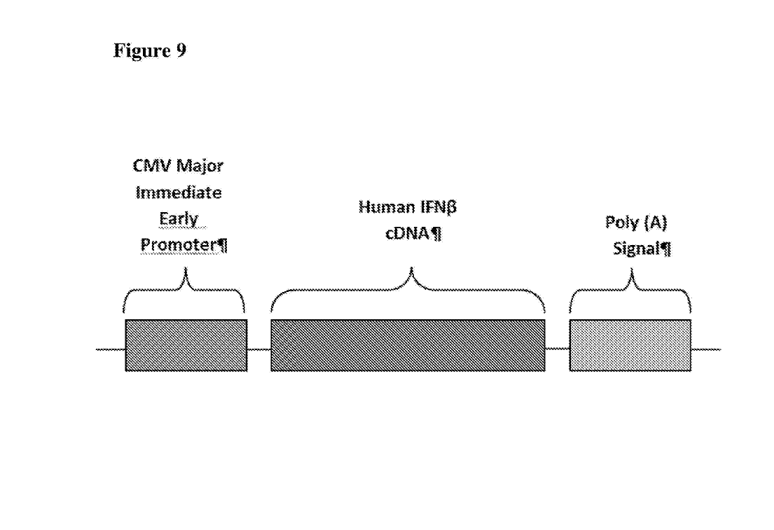

Mock infected fibroblasts pulsed with HCMV pp65 derived peptide (VTE), and CMV-IFNβ infected and rescuant infected fibroblasts were cultured in IFNβ-containing media. Clone specific CD8+ T cells were added and activation of CD8+ T cells was measured. The results are shown in FIG. 8.

The results demonstrate that CMV-IFNβ virus has a greater potential for activation of CD8+ T cells than do rescuant virus or antigen primed fibroblasts. The results further show that the increased activation does not arise solely from N as all samples contained approximately the same amount of IFNβ whereas CMV-IFNβ for example, shows at least a 4 fold increase in activation of T cells. These results clearly demonstrate the immunogenic potential of the CMV-IFNβ to invoke a polyclonal T cell response in vivo.

### Example 11

### Methodology: Construction of HCMV-IFNβ

The self-excising HCMV Merlin BAC pAL1160 (pAL1160-Merlin), that expresses an IRES-eGFP downstream of UL122 (IE2) (Stanton et al., 2010) was kindly donated by R. Stanton and G. Wilkinson (Cardiff University). Insertion of human interferon beta cDNA (Origene) was performed using two-rounds of recombineering and inserted into the Merlin 1160 BAC between the nucleotides 210325 and 210326 in a non-coding region between the HCMV genes US15 and US16. Recombineering was performed in E. coli SW102 using a SacB/KanR selection cassette. During the first round of recombineering the SacB/Kan R cassette was amplified from the plasmid pTBE100 (kindly donated by E. Mocarski) using the primers US15/16_CAS_R (5′-GAAACCCTTTTTCTCTTCTCATGGTGCGCTGCGTTCTCTGGGAGCTCGGTACCCGGGG ATC-3′) (SEQ ID No.2) and US15/16_CAS_R (5′-GATTITCGTTCGGAACTGGTTTTCGGACAGAGCAGCCGTTGAAAAGTGCCACCTGT ATGC-3′) (SEQ ID No.3). Each primer is composed of 15 nucleotides (nt) homologous to the antibiotic resistance cassette KanR/SacB at its 3′ end (underlined) and 45 nt homologous to the HCMV BAC genome either side of insertion. These primers were used to amplify the selection cassette by PCR. PCR was carried out at 94° C. for 2 mins; 5 cycles of 95° C. for 30 secs, 47° C. for 30 secs, 72° C. for 3 mins; 25 cycles of 95° C. 30 secs, 75° C. for 30 secs, 72° C. for 3 mins; 72° C. for 10 mins using the Picomaxx High Fidelity PCR System. The PCR product was treated with Dpn1 (Promega) according to manufacturer's recommendations to remove plasmid template. The digested product was purified with a Minelute Extraction Kit (Qiagen). The purified PCR product was electroporated into the pAL1160 containing SW102 cells that had been induced for 15 minutes at 42° C. and made electrocompetant. The parameters for electroporation were set at 1.75 KV, 200Ω, 25 uF in a 0.1 mm electroporation cuvette (Bio-Rad). The recombinant clones were selected at 32° C. on LB plates containing 20 mg/mL chloramphenicol (Sigma Aldrich) and 50 mg/mL kanamycin (Roche) and then characterised using PCR and restriction endonuclease profiles. The resultant BAC was designated pAL1160-Cassette. During the second round of recombineering the selection cassette was replaced with a human IFNβ cDNA cassette under the control of the strong, constitutive HCMV major immediate early (MIE) promoter. The human IFNβ cassette was amplified from the plasmid pCMV6-XL4(IFNβ) (Origene) using the primers US15/16_IFNβ_F (5′-CGTAGATGACCGTGCCATCGGTGGGTACTTGAAACCCTTTTTCTCTTCTCATG GTGCGCTGCGTTCTCTGGITGAATCAATATTGGCAATTAG-3′) (SEQ ID No.4) and US15/16_IFNβ_R (5′-CGTCAACGCCGTTGTCCACCCTCTCGCCCTAGATTTTCGTTCGGAACTGGTTTTCGGA CAGAGCAGCCGTTTAATTCAACAGGCATCTACTGAG-3′) (SEQ ID No.5). Each primer is composed of 20 nucleotides (nt) homologous to the human IFNβ cDNA cassette at its 3′ end (underlined) and 80 nt homologous to the HCMV BAC genome either side of insertion. After electroporation, the recombinant clones were selected at 32° C. on LB plates containing 20 mg/mL chloramphenicol and 7% (w/v) sucrose and then characterised using PCR and restriction endonuclease profiles. The resultant BAC was designated pAL1160-IFNβ. All constructs were verified by sequencing modified regions using Australian Genome Research Facility.

Maxiprep BAC DNA was isolated using the Qiagen Plasmid Maxi Kit according to manufacturers protocols (Qiagen) from 100 mL overnight cultures supplemented with 20 mg/mL chloramphenicol. 1×105 V5-HFFs were transfected with 2 μg maxiprep BAC DNA using a calcium phosphate mediated transfection method according to the ProFection® Mammalian Transfection System (Promega).

Virus stocks were generated in PIV5-HFFs infected with the required virus. Tissue culture supernatants were collected and stored after 100% cytopathic effect (CPE) was observed. The supernatants were centrifuged to remove cellular debris after which cell-free virus was pelleted by centrifugation at 22,000×g for 2 hours and resuspended in DMEM supplemented with 10% FCS. Viruses were titrated in triplicate by plaque assay for 10 days on PIV5-HFFs using a 1% Avicel overlay (Matrosovich et al., 2006). HFFs and PIV5-HFFs were mock infected or infected with RCMV1160-Merlin, RCMV1160-IFNβ or RCMV1160-Rescuant at an MOI of 0.01 or 3 as indicated in text, for 1.5 hours with occasional gentle rocking, after which the cells were washed with phosphate-buffered saline (PBS) and fresh media added. Plaques were visualised using a Zeiss Axiovert S100 microscope and plaque sizes determined using AxioVision Software (Carl Zeiss). Supernatant was collected 24 and 48 hours post infection and IFNβ protein expression was measured using an ELISA (PBL Assay Science)

### REFERENCES

1. Stratton, R., et al. 2000. Vaccines for the 21st Century: A Tool for Decision Making, Washington, USA, Institute of Medicine
2. Day, S., et al. 2008 J. Immunol. 180: 7158
3. Sainz, B., et al. 2005 Virol. J. 2:14
4. Stanton, R. J., et al. 2010 J. Clin. Invest. 120: 3191
5. Pass, R. F., et al. 2009 N Engl J Med 360 (12): 1191

1. A nucleic acid including:
a first region encoding:
a cytomegalovirus (CMV); or
a viral particle including a CMV protein;
a second region encoding a cytokine for preventing a CMV or viral particle translated from the first region from infecting a cell.

2. The nucleic acid of claim 1 wherein the second region is located within the first region.

3. The nucleic acid of claim 1 or 2 wherein the second region is operably linked to a promoter for enabling transcription of the second region independently of transcription of the first region.

4. The nucleic acid of claim 3 wherein the promoter enables transcription of the second region before transcription of the first region.

5. The nucleic acid of claim 3 or 4 wherein the promoter is a CMV related promoter.

6. The nucleic acid of claim 5 wherein the promoter is an MEI or IE promoter.

7. The nucleic acid of any one of the preceding claims wherein the first region includes an attenuation of a CMV gene that prevents replication of the CMV or viral particle translated from the first region in an infected cell.

8. The nucleic acid of any one of the preceding claims wherein the first region includes an attenuation that prevents infection of a bystander cell in the form a cell adjacent the infected cell, by a CMV or viral particle translated from the first region.

9. The nucleic acid of any one of the preceding claims wherein the first region includes an attenuation of a CMV gene that regulates expression of host MHC Class I or II genes.

10. The nucleic acid of any one of claim 7 to 9 wherein the attenuation is in one or more of the following genes: UL11a (viral IL-10), US2, US3, US4, US5, US6, US7, US8, US9, US10 and US11 genes.

11. The nucleic acid of any one of claims 1 to 5 wherein the first region does not include one or more of the following CMV genes: UL111a (viral IL-10), US2, US3, US4, US5, US6, US7, US8, US9, US10 and US11 genes.

12. The nucleic acid of any one of the preceding claims wherein the first region includes a mutation for modifying the immunogenicity of the CMV or viral particle encoded by the first region.

13. The nucleic acid of any one of the preceding claims wherein the first region includes one or more genes encoding a non CMV epitope.

14. The nucleic acid of any one of the preceding claims wherein the second region encodes a cytokine in the form of an interferon Type I (IFNI).

15. The nucleic acid of claim 14 wherein the IFNI is IFN-β

16. The nucleic acid of any one of the preceding claims wherein the second region encodes a further cytokine for preventing a CMV or viral particle translated from the first region from infecting a cell.

17. The nucleic acid of anyone of the preceding claims wherein the first region is linked to the second region thereby encoding a fusion protein in the form of a CMV peptide or viral particle peptide encoded by the first region that is linked to the cytokine encoded by the second region.

18. A nucleic acid or construct including:
a first region encoding CMV; and
a second region encoding IFN-β,
wherein the first region contains an attenuation of UL111a and US2, US3, US4, US5, US6, US7, US8, US9, US10, and US11.

19. A vector including a nucleic acid of any one of the preceding claims.

20. A virus or infective particle including a nucleic acid of any one of the preceding claims.

21. A cell including a nucleic acid, vector, or virus of any one of the preceding claims.

22. The cell of claim 21, further including a protein for blocking an IFN-IR or IFN-IIR signalling pathway.

23. A composition including a nucleic acid, vector, virus or cell of any one of the preceding claims.

24. A method for treating an individual having a CMV infection including the step of:
administering a nucleic acid of any one of the preceding claims to an individual having a CMV infection, thereby treating the individual for CMV infection.

25. A method for preventing an individual from infection with CMV including the step of:
administering a nucleic acid of any one of the preceding claims to an individual in whom CMV infection is to be prevented, thereby treating the individual for CMV infection.


SEQUENCE LISTING (5 sequences)

SEQ ID NO: 1 - DNA, 2110 bp, Homo sapiens
ttgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgcgaattcgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctcgactctagattgcggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaatt

SEQ ID NO: 2 - DNA, 61 bp, Cytomegalovirus
gaaaccctttttctcttctcatggtgcgctgcgttctctgggagctcggtacccggggatc

SEQ ID NO: 3 - DNA, 61 bp, Cytomegalovirus
gattttcgttcggaactggttttcggacagagcagccgtttgaaaagtgccacctgtatgc

SEQ ID NO: 4 - DNA, 93 bp, Homo sapiens
cgtagatgaccgtgccatcggtgggtacttgaaaccctttttctcttctcatggtgcgctgcgttctctggttgaatcaatattggcaattag

SEQ ID NO: 5 - DNA, 94 bp, Homo sapiens
cgtcaacgccgttgtccaccctctcgccctagattttcgttcggaactggttttcggacagagcagccgtttaattcaacaggcatctactgag